(19) United States
(12) Patent Application Publication
(10) Pub. No.: US 2022/0008632 A1
(43) Pub. Date: Jan. 13, 2022

(54) CUPPING AUXILIARY CONSUMABLE, CUPPING KIT, AND CUPPING METHOD

(71) Applicant: Wellness Allied Inc, Natick, MA (US)

(72) Inventors: Weihui Li, Natick, MA (US); Tao ZHANG, Natick, MA (US)

(21) Appl. No.: 16/926,993

(22) Filed: Jul. 13, 2020

(51) Int. Cl. A61M 1/08; A61M 1/36; A61M 1/00

(52) U.S. Cl. CPC A61M 1/08; A61M 1/0011; A61M 1/3673

(57) ABSTRACT

A cupping auxiliary consumable used in conjunction with one of the Chinese Medicine techniques, cupping. The characteristics include: a sanitary blood-absorbent pad suitable to insert into a cup during cupping treatment, and when applied, the layer against the human skin is made of a material that is permeable to blood. A cup kit for cupping is also provided, including: a cup and a sanitary blood-absorbent pad inserted into the cup. The cup can also include an adhesive surface connecting the cup and the skin; a releasing layer to protect the adhesive surface. The cupping operation can be carried out easily, the blood is absorbed into the blood-absorbent pad, thus will not soil the cup, keeping the entire cupping process hygienic.

### TECHNICAL FIELD

The present invention is related to the cupping auxiliary consumable, the cup device and the cupping method in the medical field.

### BACKGROUND

Body's circulation is responsible to send blood, oxygen and nutrients to all the cells throughout the body. When blood flow to a specific part of the body slows down, the body will experience symptoms of poor circulations. The common symptoms of poor circulation include: tingling, numbness, throbbing or stinging pain, muscle cramps.

The causes or consequences of poor circulation can be peripheral artery diseases, blood clots, varicose veins, diabetes, obesity, Raynaud's disease etc.

There have been different methods to diagnose poor circulation: 1) physical exam to detect pain and swelling; 2) blood antibody test to detect inflammatory conditions, such as Raynaud's disease; 3) blood sugar test for diabetes; 4) blood test to look for high levels of D dimer in the case of a blood clot; 5) ultrasound or CT scan; 6) blood pressure tests of the arms and legs.

Treatment for poor circulation may include: 1) compression socks for painful or swollen legs; 2) laser or endoscopic vein surgery for varicose veins; 3) insulin for diabetes; 4) special exercise program recommended by doctors; 5) blood thinner drugs such as aspirin.

Besides, there are another type of treatment that hasn't been explored in conventional medicine yet, but was adopted in many countries in the past: bloodletting.

Bloodletting has been criticized widely mainly because: 1) it caused some death in the past, including President George Washington, who had been bled heavily after he developed a throat infection, leading to too much blood loss and blood pressure drop; 2) some people were impressed that bloodletting is done with leeches, which is ethically and hygienically inappropriate. These biased media exposures prevent the adoption of bloodletting in the medical field.

In fact, bloodletting has been undervalued. In western world, one of the most influential medical journals, the Lancet, got its name exactly because of the effectiveness of bloodletting with lancet. In Traditional Chinese Medicine, bloodletting was documented more than two thousand years ago. It is an effective way to promote circulation. However, instead of bleeding big veins or arteries, Traditional Chinese Medicine usually do bloodletting on spider veins with lancets for only a few drops of blood.

In Traditional Chinese Medicine, there is another therapy called cupping. It makes use of negative pressure to suck on the skin. The negative pressure can be created using fire to heat the air inside the cup (FIG. 1A) or using a vacuum pump to pump out the air (FIG. 1B).

When the cupping is performed after puncturing the skin with lancet, some blood will be sucked out. This is often called bloodletting cupping in Chinese Medicine. With this type of bloodletting, at most a few milliliter blood can be sucked out. When only a few drops of blood are sucked out, they are usually wiped away with cotton balls. If more blood is sucked out, the medical staff usually uses bigger gauze to absorb the blood while removing the cup from the skin.

FIG. 1 shows common application scenarios of fire cupping, air-pump cupping, and bloodletting cupping. 110 in FIG. 1A is a burning cotton, which is used to heat the air in the glass cup 120 and expands the air. Then the cup is quickly covered on the skin. When the air inside the cup cools down again, the pressure inside the cup gets smaller, making the cup suck on the skin. The air-pump cupping cup in FIG. 1B is composed of an air-pumping valve 130, the cup wall 140 and the suck opening 150. The negative pressure is generated by the suction gun 155. If the skin is punctured with a sharp needle before cupping, blood 160 can be sucked out during the cupping process (FIG. 1C).

### SUMMARY

According to an aspect of the present invention, a cupping auxiliary consumable is provided. The characteristics includes: a blood-absorbent pad suitable to place into a cupping cup with the surface against the skin being made of material that is permeable to body fluid.

Preferably, the size of the blood-absorbent pad is suitable for being placed inside the cup, and the shape of the pad is flat or concave.

Preferably, the surface layer of the blood-absorbent pad against the skin is treated with anticoagulant substances to prevent the blood clot and promote bloodletting during the cupping treatment.

Preferably, the backing layer of the blood-absorbent pad is impermeable to fluid, to prevent the absorbed body fluid from leaking out to pollute the environment or spread diseases.

Preferably, an adhesive is coated around the blood-absorbent pad to stick to the cup.

Preferably, the interior of the blood-absorbent pad is cellulose material, polymeric foam, or absorbent resin.

Preferably, the cupping auxiliary consumables further include: an adhesive layer to adhere the cupping cup to the skin; and a single-sided release paper for protecting the adhesive layer before cupping and seal the soiled blood-absorbent pad after cupping.

Preferably, the adhesive layer is a double-sided adhesive tape. When applied, one side of it adheres to the edge of the cup and the other side of it adheres to the skin.

Preferably, the release paper is impermeable to liquid with one side being coated with release agent such as silicon oil, and the other side being the plain paper; before cupping, the release paper can be easily removed from the adhesive tape, and after cupping, the plain paper side is placed back to the adhesive tape to seal the soiled pad inside.

Preferably, the adhesive tape and the blood-absorbent pad are integrated, or the two are separable.

According to another aspect of the present invention, a cup kit for bloodletting cupping is provided, including: a cup; a sanitary blood-absorbent pad placed in the cup.

Preferably, the cup further includes: an adhesive surface adhering the cup to the skin; a single-sided release paper that protects the adhesive surface before use and seal the soiled blood-absorbent pad after use.

Preferably, the adhesive surface is integrated with the cup, and the entire cup is disposable.

Optionally, the cup has at least one suck opening, which is in contact with the skin during application to suck on the skin due to the internal low pressure.

Optionally, the material of the cup is glass, metal, silicone, plastic or rubber.

Optionally, the cup also includes a mechanical structure that can be operated to create low pressure inside the cup.

Optionally, the mechanical structure is an air suction port, from which air can be suctioned out and internal low pressure is created.

Alternatively, the cup can be operated to reduce the internal space by external force, followed by attaching the cup suck opening to the skin, increasing the internal space back by the elasticity of the cup or external force to create internal low pressure.

According to another aspect of the present invention, a method for bloodletting cupping with a cup kit is provided. The cup kit includes a cup, a sanitary blood-absorbent pad placed inside the cup, an adhesive surface around the cup suck opening for connecting the cup and the skin, and a release paper. The method includes: removing the release paper from the adhesive surface; attach the cup to the skin; create a low pressure inside the cup to suck on the human skin; the blood is sucked out of the skin and absorbed into the blood-absorbent pad; releasing the low pressure; remove the cup from the skin; discard the entire cup kit if it is disposable, or take the blood-absorbent pad away from the cup to discard and recycle the cup for sterilization.

Preferably, in the cupping method, the release paper is impermeable to fluid with one sided being coated with release agent such as silicon oil, and the other side being plain paper. Before the cupping, the surface with release agent is attached to the adhesive tape. When the release paper is removed from the adhesive tape. After the cupping, place back the release paper onto the adhesive tape on the plain paper side to seal the soiled pad.

Using the cupping auxiliary consumables, the cup kit and the bloodletting cupping method of the embodiments of the present invention, by placing a blood-absorbent pad in the cup, the blood sucked out from the human body is directly absorbed by the blood-absorbent pad and stays there, without the need for the medical staff to spare the hands to wipe out the blood with gauze pads. The doctor can carry out the bloodletting cupping operation with less hassle, and with the blood sucked away by the blood-absorbent pad without soiling the cup, the whole bloodletting cupping becomes a sanitary process and minimizes infection possibilities.

### BRIEF DESCRIPTION OF THE DRAWINGS

FIGS. 1A-1C shows the appearance and components of the common existing cupping devices and use scenario.

FIG. 2A-2D are schematic diagrams of a blood-absorbent pad according to an embodiment of the present invention and the use of a double-sided adhesive tape to adhere the cup to human skin.

FIGS. 3A-3C and 4A-4C show an example where the double-sided tape and the blood-absorbent pad are individual parts.

FIG. 5 shows an appropriate application of the adhesive surface and release paper.

FIG. 6 shows a structural view of a cup that can be used in conjunction with the blood-absorbent pad of the present disclosure.

FIG. 7 shows a structural view of another cup that can be used in conjunction with the blood-absorbent pad of the present disclosure.

FIG. 8 is a structural diagram of another cup that can be used in conjunction with the blood-absorbent pad of the present disclosure.

FIG. 9 is a structural diagram of another cup that can be used in conjunction with the blood-absorbent pad of the present disclosure.

FIG. 10 is a structural diagram of another cup that can be used in conjunction with the blood-absorbent pad of the present disclosure.

FIG. 11 shows a structural view of another cup that can be used in conjunction with the blood-absorbent pad of the present disclosure.

### DETAILED DESCRIPTION

Hereinafter, exemplary embodiments according to the present application will be described in detail with reference to the drawings. Obviously, the described embodiments are only a part of the embodiments of the present application, not all the embodiments of the present application, and it should be understood that the present application is not limited by the example embodiments described herein.

The following describes an example of a cupping auxiliary consumable and a cup kit equipped with such a cupping auxiliary consumable for bloodletting cupping.

The present application provides a blood-absorbent pad suitable for being placed in a cup for bloodletting cupping. The surface layer of the blood-absorbent pad that is adjacent to the human skin is made of a material permeable to fluid.

The blood-absorbent pad can be sold by itself, and the shape and size can be adjusted according to the size and shape of the cups on the market. Or the blood-absorbent pad and the cup can pre-assembled to form a disposable cup kit.

The blood-absorbent pad for cupping has the following benefits: during cupping, the blood sucked out by the cupping is directly absorbed by the blood-absorbent pad, therefore there is no need for the medical staff to spare the hands to wipe the blood away with gauzes or the like. Furthermore, it will not soil the cup and make the whole operation process hygiene.

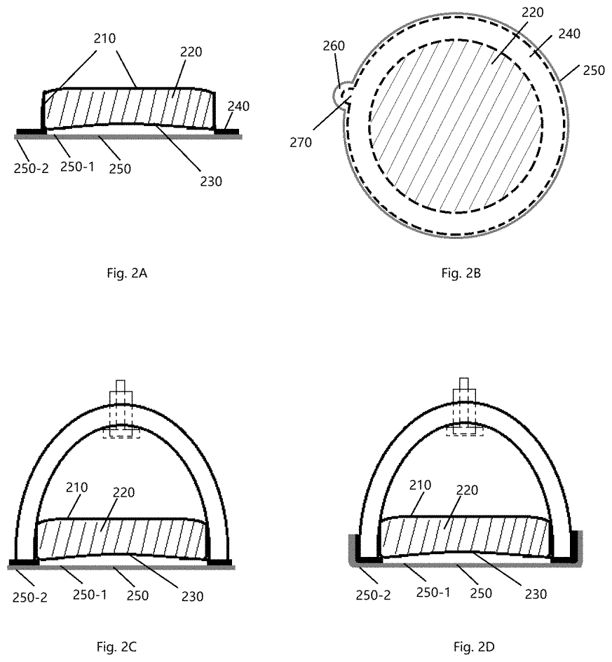

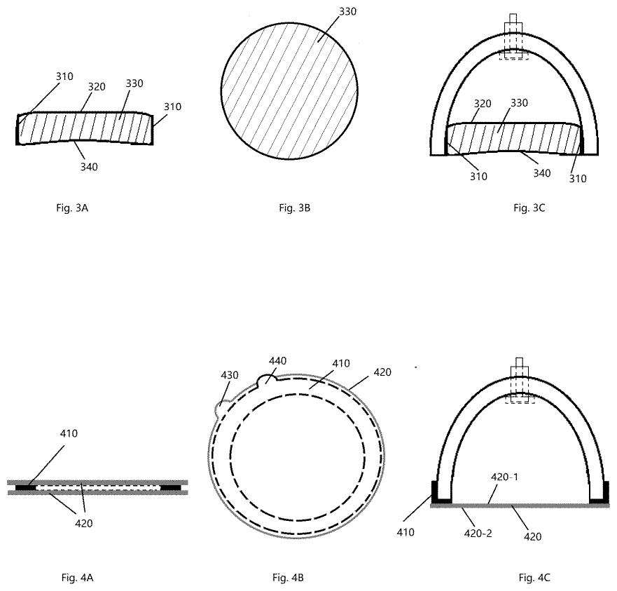

FIGS. 2A-2D show schematic diagrams of a blood-absorbent pad according to an embodiment of the present invention and the use of double-sided adhesive tape to connect the cup and the skin.

FIG. 2A is a side view of a blood-absorbent pad. The blood-absorbent pad 220 is a material that absorbs human body fluids (including blood) and is not easy to be squeezed out again. The sides and backing of the blood-absorbent pad 210 are impermeable to human fluids (including blood)) to prevent the absorbed body fluid from leaking out and polluting the environment or spreading diseases. The surface layer 230 of the blood-absorbent pad 220 is permeable to body fluid through which the body fluid is absorbed in the pad, and it may be treated with anti-coagulant substance to prevent blood clotting and promote better bloodletting. The inside of the blood-absorbent pad may be cellulose material, polymeric foam, or absorbent resin.

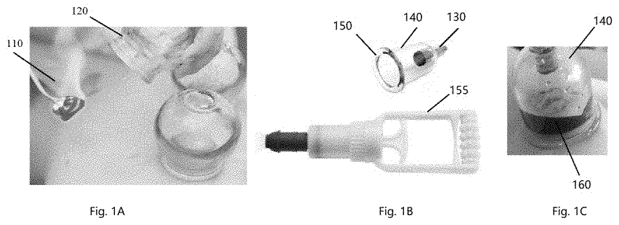

240 in FIGS. 2A-2D is double-sided tape. When in use, one side of the tape adheres to the skin and the other side adheres to the suck opening of the cup (as indicated by 150 in FIG. 1B). 250 is single-sided release paper, with one side being coated with a release agent 250-1, such as silicon oil, and the other side the plain paper 250-2. Before use, the single-sided release paper 250 covers both sides of the double-sided tape 240 to prevent it from sticking to other objects, and it is easy to peel off during use. When in use, put the blood-absorbent pad into the jar, and peel off the release paper on both sides of the double-sided tape. One side adheres to the cup, and the other side adheres to the skin, so that the cup does not get off the skin easily during the cupping process. After the cupping treatment, take the cup away from the skin together with the blood-absorbent pad, and put the plain paper side 250-2 of the release paper back on the double-sided tape, which can effectively seal the blood-absorbent pad with body fluid absorbed and prevent it from contaminating others. If it is a disposable cup, then the whole cup can be discarded with the blood-absorbent pad. If the cup is reusable, the sealed blood-absorbent pad is taken apart from the cup and discarded. In this embodiment, the blood-absorbent pad 220 and double-sided tape 240 are an integrated piece.

FIG. 2B shows a top view of a blood-absorbent pad with double-sided tape. 260 is a hand grab piece, with which release paper can be easily peel off from the double-sided tape.

FIG. 2C is a schematic diagram showing the use case of the blood-absorbent pad in a cup. The side of the blood-absorbent pad against the skin may be treated with anticoagulant agent to prevent blood clotting during the bloodletting treatment and promote the removal of blood stasis.

FIG. 2D is an alternative schematic diagram with the double-sided tape and release paper being a little bigger so that they can be folded upwards to the outside of the cup. The advantage of this design is that, that extra double-sided tape on the side of the cup will remain sticky after the treatment and allow the plain paper side of the release paper to seal the soiled blood-absorbent pad.

It should be noted that the double-sided adhesive tape and the blood-absorbent pad may be integrated, or the two may be separated.

FIGS. 3A-3C and 4A-4C show the case where the double-sided adhesive tape and the blood-absorbent pad are separated.

In FIGS. 3A-3C, the blood-absorbent pad 330 is a material that absorbs body fluids (including blood) and cannot be easily squeezed out body fluid again. The backing layer 320 of the blood-absorbent pad is impermeable to liquid, and the surface layer 340, which attaches to skin when in use, is permeable to human body fluids. The surrounding surface 310 of the blood-absorbent pad is coded with adhesive to adhere to the inner wall of the cup when in use.

FIGS. 4A-4C shows a double-sided tape 410 and a single-sided release paper 420. The double-sided tape 410 is ring-shaped, and when in use: one side adheres to the opening of the cup and the other side adheres to the skin. The single-sided release paper 420 has one smooth side 420-1 being coated with release agent and one plain paper side 420-2. Before application, the double-sided tape 410 is covered by release paper in both sides. When is use, remove the release paper from one side of the double-sided tape, stick the double-sided tape to the opening of the cup. Then the other side of release paper is removed and the cup is put on the human body to do the cupping. The release paper is put aside for late use. After cupping, the cup is taking away from skin together with the double-sided cup and the plain paper side of the release paper is stuck back to the double-sided tape. As shown in FIG. 4C, the ring-shaped double-sided tape is a little bigger than the cup opening so that the double-sided tape can be folded to the outside surface of the cup. This will leave that folded tape untouched during cupping treatment and remaining sticky for the release paper to seal the soiled blood-absorbent pad. The release paper can also have one hand grab piece 430 for hand to peel off or put back the release paper.

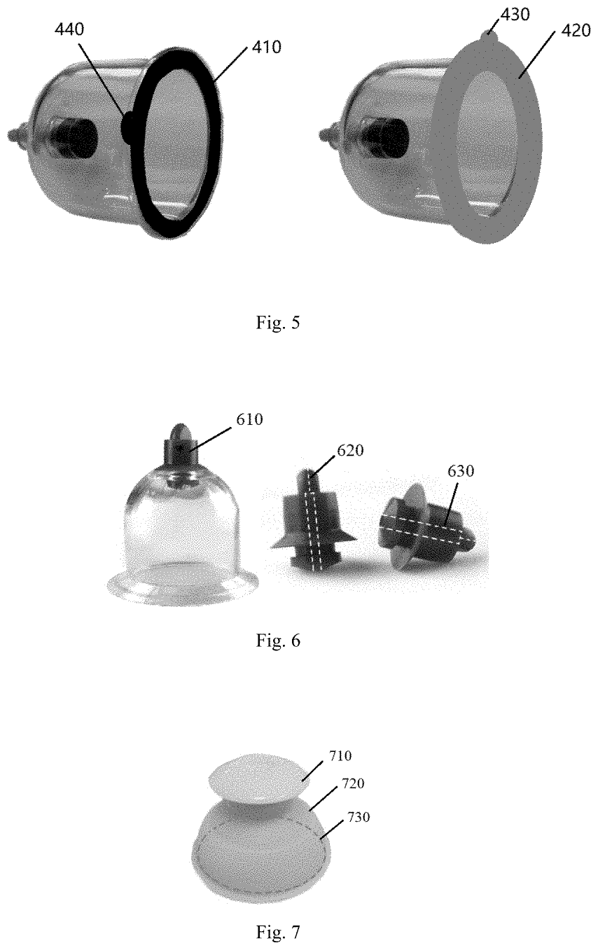

FIG. 5 shows a schematic diagram of an integrated kit of cup and double-sided tape with a release paper according to an embodiment of the present invention. The opening of the cup is pre-covered with double-sided tape (as shown in FIGS. 4A-4C at 410), release paper (shown at 420 in FIGS. 4A-4C) and a grab piece (shown at 430 in FIGS. 4A-4C). When the separate blood-absorbent pad shown in FIGS. 3A-3C and the integrated kit of cup and double-sided tape shown in FIG. 5 are used together, the function is same as the one shown in FIG. 2C.

The blood-absorbent pad can be sold alone, and the shape and size can be adjusted according to the size and shape of the cup in the market. Or the blood-absorbent pad can be integrated with the cup to form a disposable cup for bloodletting.

The following will describe different cups that can be used for the cup kit according to the embodiment of the present invention.

The cup used in the embodiment of the present invention may be a cup as shown in FIG. 6. The cup includes a lower cup wall and an upper rubber plug 610. The cup has two openings. The upper opening connects with the outside through a cylinder channel 630 and a cut 620 on the tip of the rubber plug. The lower opening is the opening where the cup contacts with the human skin. When pumping air out of the cup with a suction gun, the cut will open due to the pressure difference between the air in the cup and the air in the suction gun chamber. When stop pumping, the atmosphere air pressure is bigger than the pressure inside the cup so the cut 620 is pressed to close, preventing the air to leak into the cup.

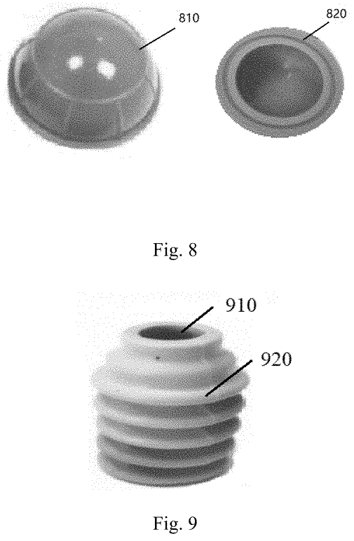

The cup can also have one opening, for example, as shown in FIGS. 7-9. The cup shown in FIG. 7 includes a handle 710, a middle portion 720 forming a cavity inside, and a suck opening 730 to suck on the skin. When in use, the general principle is as the following: squeeze the cup firmly to minimize the internal cavity space; securely seal the suck opening 730 on the skin; let the cup recover its original shape through its own rebound force. Through the above three steps, a low pressure is formed inside the cavity, allowing the cup to suck on the skin. The cup of FIG. 8 also has only one opening portion 820 and cup wall 810. The working principle is the same as FIG. 7. FIG. 9 also uses a similar principle, except that the shape of the suck opening 910 and the cup wall 920 are different.

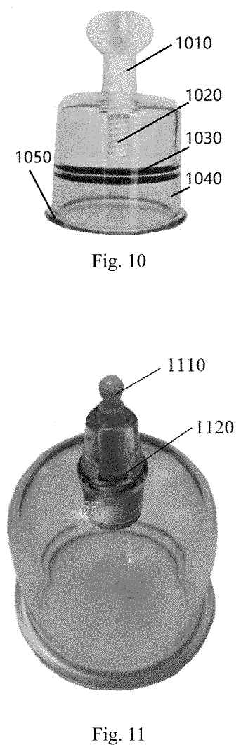

FIG. 10 shows another example of a cup. It includes a knob 1010, a rotating rod 1020, a sealed disc 1030 connected to the rotating rod, the cup wall 1040, and the suck opening 1050. The working principle is as following: the disc 1030 starts at a position close to the suck opening 1050. The suck opening firmly covers on the skin, rotate the knob 1010 to lift the disk through the rotation rod 1020. Through the above steps, the internal cavity between the skin and the disc becomes larger and the pressure smaller, so that the cup creates a pulling force to suck on the skin.

The cup can also be as shown in FIG. 1B and FIG. 11. The cup shown in FIG. 1B includes a suck opening 130, a cap wall 140, and an adsorption port 150. FIG. 11 is the same cup as shown in FIG. 1B except that FIG. 11 shows more details about how it works. The cup has a movable stopper 1110 and a sealing ring 1120; when pumping, outward gas will cause the stopper 1110 to move outward, separating it from the sealing ring, so that the air is pumped away from the cup, forming an internal low pressure. When stop the pumping, the external high pressure will press the stopper back towards the sealing ring to prevent air leakage.

The present disclosure provides a method for cupping with a blood-absorbent pad, double-sided adhesive and a cup according to an embodiment of the present invention: peeling the release paper off the double-sided adhesive; connect the cup and the skin; form a low pressure inside the cup thereby sucking up human skin; human blood is sucked from the skin to the blood-absorbent pad; low pressure is removed; take the cup away from the skin; seal the soiled blood-absorbent pad with release paper on the plain paper side.

The embodiments of the present invention have been described above. The above description is exemplary, not exhaustive, and is not limited to the disclosed embodiments. Many modifications and variations will be apparent to those of ordinary skill in the art without departing from the scope and spirit of the illustrated embodiments. Therefore, the protection scope of the present invention should be subject to the protection scope of the claims.

1. A cupping kit for the bloodletting cupping technique, including:
a cupping cup;
a blood-absorbent pad inserted in the cap.

2. The cupping kit of claim 1, further comprising:
an adhesive to adhere the cup to the skin;
a release paper on top of the adhesive.

3. The cupping cup of claim 2, the adhesive is an integrative part of the cup and the entire cup is disposable.

4. The cupping cup of claim 1, wherein the cup has at least one opening, which is in contact with the skin during application, and the skin can be sucked up by creating low pressure inside the cup.

5. The cupping cup of claim 1, the material of the cup is glass, metal, silicone, plastic, or rubber.

6. The cupping cup of claim 1, further comprising a mechanical structure that can be operated to create a low pressure inside the cup.

7. The cupping cup of claim 1, the mechanical structure is a suction port, through which the internal low pressure is created.

8. The cup of claim 1, the cupping cup is operable to reduce the internal space first through external force, and then the internal space is increased through the cup's elasticity or external force, thereby forming a low pressure.

9. A bloodletting cupping method with a cupping kit, which includes a cupping cup, a sanitary blood-absorbent pad inserted into the cup, an adhesive used to connect the cup and skin, and a release paper to protect the adhesive, the method including:
puncturing the skin with a sharp;
removing the release paper;
connecting the cup and the skin through the adhesive;
creating a low pressure inside the cup, thereby sucking up human skin;
absorbing the blood that is sucked out of the skin;
releasing the low pressure;
removing the cup from the skin;
discarding the entire cup if it is disposable, or removing the blood-absorbent pad from the cup and discarding the pad, recycling the cup for sterilization.

10. A cupping auxiliary consumable used in conjunction with a cupping cup, includes:
a blood-absorbent pad that is suitable to insert into the cup during cupping treatment, with the surface against the human skin being made of a material that is permeable to blood.

11. The cupping auxiliary consumable according to claim 10, wherein
size of the blood-absorbent pad is suitable to place inside the cup, and surface against the human skin is flat or curved in a concave shape.

12. The cupping auxiliary consumable according to claim 10, wherein the surface against human skin has been treated with anticoagulant substances.

13. The cupping auxiliary consumable according to claim 10, the backing of the blood-absorbent pad is impermeable to fluid, to prevent absorbed body fluid from leaking out to pollute environment or spread diseases.

14. The cupping auxiliary consumable as claimed in claim 10, an adhesive is coated around the blood-absorbent pad to stick to the cup.

15. The cupping auxiliary consumable of claim 10, wherein the interior of the blood-absorbent pad is cellulose material, polymeric foam, or absorbent resin.

16. The cupping auxiliary consumable as claimed in claim 10, further comprising:
an adhesive to attach the cupping cup to the patient's skin;
a release paper on top of the adhesive material.

17. The cupping auxiliary consumables according to claim 16, the adhesive material is double-sided tape, when in using, one side adhering to the patient's skin and the other side adhering to the cupping cup.

18. The cupping auxiliary consumable of claim 16, the release paper is impermeable to liquid with one side being coated with release agent such as silicon oil, and the other side being the plain paper; before cupping, the release paper can be easily removed from the adhesive, and after cupping, the plain paper side is placed back to the adhesive to seal the contaminated pad inside.

19. The cupping auxiliary consumable of claim 16, wherein the adhesive is an integrated part of the blood-absorbent pad, or the adhesive is a separate part from the blood-absorbent pad.

20. The cupping auxiliary consumable of claim 10, wherein the blood-absorbent pad includes a layer against the skin, a backing layer and a blood-absorbent layer enclosed between.
